(19) United States
(12) Patent Application Publication
(10) Pub. No.: US 2014/0170743 A1
(43) Pub. Date: Jun. 19, 2014

(54) PHOTOBIOREACTOR FOR CULTURING MICROALGAE

(71) Applicant: HYUNDAI MOTOR COMPANY, Seoul (KR)

(72) Inventor: Dae-Young Goh, Suwon (KR)

(73) Assignee: HYUNDAI MOTOR COMPANY, Seoul (KR)

(21) Appl. No.: 13/858,318

(22) Filed: Apr. 8, 2013

(30) Foreign Application Priority Data
Dec. 17, 2012 (KR) 10-2012-0147253

(51) Int. Cl. C12M 1/00

(52) U.S. Cl. CPC C12M 21/02
USPC 435/292.1

(57) ABSTRACT

Disclosed is a structure of a photobioreactor for culturing microalgae capable of enhancing carbon dioxide (CO₂) fixation efficiency by microalgae and simultaneously saving installation cost by forming a sealing structure in a microalgae growth chamber using a roller and a hydraulic pressure. More particularly, the present invention relates to a photobioreactor for culturing microalgae including a culture water bath configured to store culture water containing microalgae and feed the culture water with carbon dioxide (CO₂), an aquatic plant formed on at least one side of the culture water bath to store water (H₂O), and a lid configured to cover an upper portion of the culture water bath. Further, a watercourse is formed below the culture water bath and the aquatic plant.

### CROSS-REFERENCE TO RELATED APPLICATION

This application claims under 35 U.S.C. §119(a) the benefit of Korean Patent Application No. 10-2012-0147253 filed on Dec. 17, 2012, the entire contents of which are incorporated herein by reference.

### BACKGROUND

(a) Technical Field

The present disclosure relates to a structure of a photobioreactor for culturing microalgae capable of enhancing carbon dioxide (CO₂) fixation efficiency by microalgae. Further provided is a photobioreactor structure that further reduces installation costs by forming a sealing structure in a microalgae growth chamber using a roller and hydraulic pressure.

(b) Background Art

With the advent of environmental issues, such as global warming and exhaustion of fossil fuel, there have been various attempts to solve these environmental issues all over the world. Among these attempts, a biological CO₂ reduction technology has been used to fix carbon dioxide (CO₂) and produce biodiesel using a photosynthetic action of microalgae. This technology can be carried out under normal temperature/pressure conditions, and thus has an advantage in that it can use the principle of the carbon cycle in the natural world. Therefore, the biological CO₂ reduction technology has been considered to be the most practical alternative to reduce greenhouse gases.

Also, microalgae can serve to reduce waste disposal problems and fix carbon dioxide (CO₂) due to their various abilities. Such microalgae has been used to produce fuel materials, cosmetics, a fodder, food colorings, and other desired materials such as medicinal source materials. As a result, their applications have increased with continuous finding of desired higher value-added materials.

A photobioreactor has been used to perform a photobiological reaction of the microalgae. In order to perform effective design of a photobioreactor for culturing high-concentration microalgae, it is necessary to develop a photobioreactor which can employ light, maintain components in a medium, screen the species having an excellent ability to absorb carbon dioxide (CO₂), and culture the microalgae in a large scale.

In general, the photobioreactors for culturing microalgae may be mainly divided into open pond systems for culturing microalgae outdoors, and closed systems using a closed reactor.

The open pond system has an advantage in that the initial investment cost is very low since it is installed in open watercourses or ponds. However, this system has problems in that a large installation space is required due to the low productivity per unit volume, and carbon dioxide (CO₂) is discharged into the atmosphere without fixation during input of the collected carbon dioxide (CO₂).

The closed system, a representative of which is a tubular reactor, has an advantage in that microalgae can be grown to a high density in a small-sized closed system since it has a CO₂ sealing structure. However, such a system has a problem in that the installation cost is very high due to its complex structure as compared with the open pond systems.

Therefore, there is a demand for a microalgae culture system in which a new CO₂ sealing structure is applied to a watercourse-type microalgae growth chamber having a low installation cost so as to enhance the CO₂ fixation efficiency by microalgae and so as to provide investment and economic feasibility as well.

### SUMMARY OF THE DISCLOSURE

The present invention provides a photobioreactor for culturing microalgae capable of improving the carbon dioxide (CO₂) fixation efficiency as compared with a conventional watercourse-type culture system, and further capable of providing installation cost savings as compared with a conventional tubular culture system.

The technical problems to be solved in the present invention are not limited to the above-described technical problems, and thus it should be understood that technical problems which are not described in this specification will be made apparent from the detailed description of the invention by those skilled in the art.

According to one aspect, the present invention provides a photobioreactor for culturing microalgae, wherein the photobioreactor includes a culture water bath configured to store culture water containing microalgae and feed the culture water with carbon dioxide (CO₂), an aquatic plant formed on at least one side of the culture water bath to store water (H₂O), and a lid configured to cover an upper portion of the culture water bath. According to various embodiments, a watercourse is formed below the culture water bath and the aquatic plant.

According to various embodiments of the present invention, the lid is formed of polycarbonate.

According to various embodiments of the present invention, the lid extends along a wall frame forming the culture water bath.

According to various embodiments of the present invention, at least one roller is formed on a side of the culture water bath to closely attach the lid to the culture water bath. The roller may be closely attached to one side of the culture water bath to closely attach the lid to the culture water bath by means of a rotary motion.

According to various embodiments of the present invention, the watercourse is formed of cement. In this case, a surface of the watercourse may be finished with fiber-reinforced plastics (FRP).

According to various embodiments of the present invention, the carbon dioxide (CO₂) present in the culture water bath is hermetically sealed under a hydraulic pressure by adjusting a hydraulic level of water in the aquatic plant so that the hydraulic level of water in the aquatic plant can be set to a level higher than a hydraulic level of the culture water in the culture water bath.

According to various embodiments of the present invention, the oxygen (O₂) formed in the culture water bath is discharged out of the culture water bath by adjusting a hydraulic level of water in the aquatic plant so that the hydraulic level of water in the aquatic plant can be set to a level lower than the hydraulic level of the culture water in the culture water bath. Other features and aspects of the present invention will be apparent from the following detailed description, drawings and claims.

### BRIEF DESCRIPTION OF THE DRAWINGS

The above and other features of the present invention will now be described in detail with reference to certain exemplary embodiments thereof illustrated the accompanying drawings which are given hereinbelow by way of illustration only, and thus are not limitative of the present invention, and wherein:

FIGS. 1 and 2 are exemplary diagrams illustrating a structure of a photobioreactor for culturing microalgae as known in the related art;

FIG. 3 is a configuration diagram illustrating a photobioreactor for culturing microalgae according to one exemplary embodiment of the present invention; and

FIGS. 4 and 5 are exemplary diagrams illustrating a CO₂ sealing structure and an O₂ discharging structure of the photobioreactor for culturing microalgae according to one exemplary embodiment of the present invention.

It should be understood that the appended drawings are not necessarily to scale, presenting a somewhat simplified representation of various preferred features illustrative of the basic principles of the invention. The specific design features of the present invention as disclosed herein, including, for example, specific dimensions, orientations, locations, and shapes will be determined in part by the particular intended application and use environment.

In the figures, reference numbers refer to the same or equivalent parts of the present invention throughout the several figures of the drawing.

### DETAILED DESCRIPTION

Hereinafter reference will now be made in detail to various embodiments of the present invention, examples of which are illustrated in the accompanying drawings and described below.

Prior to the description, it should be understood that the terminology used in the specification and appended claims should not be construed as limited to general and dictionary meanings, but interpreted based on the meanings and concepts corresponding to technical aspects of the present invention on the basis of the principle that the present inventors are allowed to define the terms appropriately for the best explanation. Therefore, the description proposed herein is just a preferable example for the purpose of illustrations only, not intended to limit the scope of the invention, so it should be understood that other equivalents and modifications could be made thereto without departing from the scope of the invention.

It is understood that the term “vehicle” or “vehicular” or other similar term as used herein is inclusive of motor vehicles in general such as passenger automobiles including sports utility vehicles (SUV), buses, trucks, various commercial vehicles, watercraft including a variety of boats and ships, aircraft, and the like, and includes hybrid vehicles, electric vehicles, plug-in hybrid electric vehicles, hydrogen-powered vehicles and other alternative fuel vehicles (e.g. fuels derived from resources other than petroleum). As referred to herein, a hybrid vehicle is a vehicle that has two or more sources of power, for example both gasoline-powered and electric-powered vehicles.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about”.

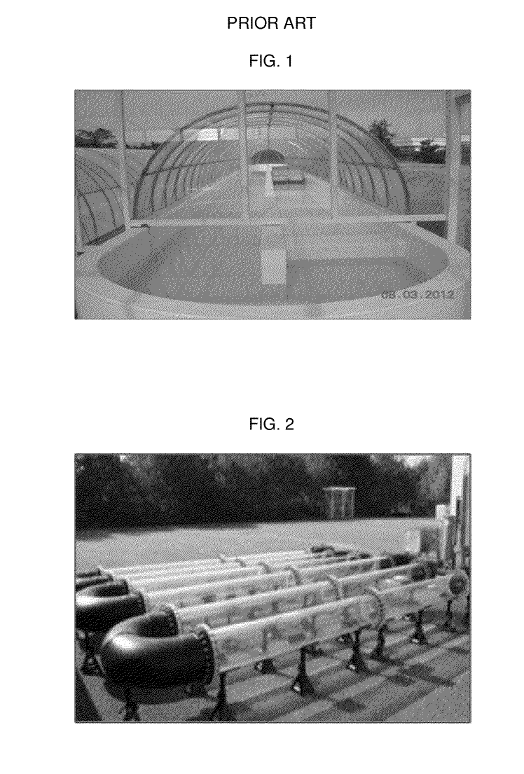

FIGS. 1 and 2 are exemplary diagrams illustrating a structure of a photobioreactor for culturing microalgae as known in the related art.

The conventional photobioreactor for culturing microalgae includes three parts: a nutrient feeding unit, a microalgae photobioreactor and a microalgae harvesting unit.

The nutrient feeding unit serves to feed nutrients and water required for growth of microalgae. The microalgae photobioreactor serves to fix carbon dioxide (CO₂) by allowing the microalgae to perform a photosynthetic action using a light source such as natural light/artificial light. The microalgae harvesting unit serves to separate the grown microalgae.

The microalgae photobioreactor is a device that actually fixes carbon dioxide (CO₂) regardless of the nutrient feeding unit, and thus is a core part of a biological CO₂ fixing unit. In the microalgae photobioreactor, the microalgae uses dissolved carbon dioxide (CO₂) and a light source to perform a photosynthetic action, thereby biologically fixing carbon dioxide (CO₂) to produce a microalgae biomass.

The microalgae biomass is subjected to a process, such as lipid extraction or saccharification, and is then used as a source for desired materials such as biodiesel and glucose.

The microalgae photobioreactor receives collected carbon dioxide (CO₂) to biologically convert the carbon dioxide (CO₂) through photosynthesis of the microalgae. In this case, the fact that carbon dioxide (CO₂) has low solubility in water and a reaction velocity of photosynthesis, which is a slow biological process, should be taken into consideration.

These conventional microalgae photobioreactors may be divided into an open pond system and a closed system using a closed reactor.

Referring to FIG. 1, a configuration of the open pond system is shown. Here, it can be shown that an upper portion of the culture water bath is configured to store microalgae and culture water and comes in contact with the atmosphere. That is, the open pond system has to discharge most of the carbon dioxide (CO₂) injected into the open pond system since the culture water bath does not have a closed structure, which leads to a decrease in CO₂ fixation efficiency.

Referring to FIG. 2, a configuration of the closed system is shown. Here, it can be shown that the culture water bath configured to store microalgae and culture water is isolated from the atmosphere to form a closed structure. Although the closed system exhibits high CO₂ fixation efficiency due to the sealing structure of the culture water bath, it has a problem in that the culture water bath is formed with a complete sealed structure, which leads to an excessive increase in the installation cost.

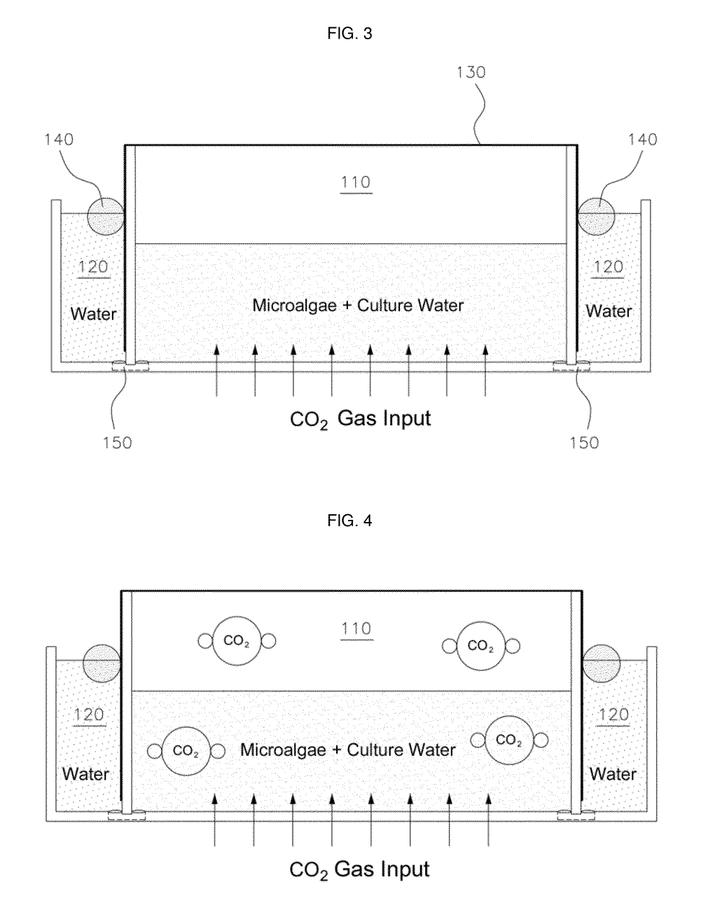

FIG. 3 is a configuration diagram illustrating a photobioreactor for culturing microalgae according to one exemplary embodiment of the present invention.

As shown, the photobioreactor for culturing microalgae according to the exemplary embodiment includes a culture water bath 110 configured to store culture water containing microalgae and feed the culture water with carbon dioxide (CO₂), an aquatic plant 120 formed on at least one side of the culture water bath to store water (H₂O), and a lid 130 configured to cover upper portions of the culture water bath 110 and the aquatic plant 120.

The culture water bath 110 is configured to store the microalgae and the culture water, and may be fed with carbon dioxide (CO₂) for photosynthesis of the microalgae. For this purpose, the culture water bath 110 may be provided with a plurality of carbon dioxide input ports (not shown) or any other suitable means for introducing the carbon dioxide. The culture water bath 110 may be formed using any known materials, such as cement.

Algae favorable for CO₂ fixation, such as chlorophyll-a or blue-green algae, may be used as the microalgae.

The aquatic plant 120 may be formed on at least one side of the culture water bath 110. In this case, carbon dioxide (CO₂) may be hermetically sealed under a hydraulic pressure, or oxygen (O₂) may be discharged by adjusting a hydraulic level of water stored in the aquatic plant 120.

The wall frame forming the aquatic plant 120 may be formed at a height lower than the wall frame forming the culture water bath 110. This can facilitate installation of a roller 140 which will be described later, and close attachment of the lid 130 through rotation of the roller 140.

The lid 130 is formed to cover an upper portion of the culture water bath 110, and is formed to extend along the wall frame of the culture water bath 110. The lid 130 serves to prevent the carbon dioxide (CO₂) input into the culture water bath 110 from flowing into the atmosphere.

In order to prevent the outflow of the carbon dioxide (CO₂) from the culture water bath 110 into the atmosphere as described above, the lid 130 should be completely attached to the wall frame of the culture water bath 110 so that the carbon dioxide (CO₂) cannot be leaked from an upper portion of the culture water bath 110.

For this purpose, according to the present invention, at least one roller 140 may be provided on one side of the culture water bath 110 to closely attach the lid 130 to the culture water bath 110.

The roller 140 is closely attached to one side of the culture water bath 110, and thus serves to pull and push the lid 130 using a rotary motion, thereby closely attaching the lid 130 to the culture water bath 110.

Meanwhile, there are many factors, such as compositions of a medium, temperature, pH, light intensity, and intensity of radiation, which affect an increase in fresh algae weight and desired products of the microalgae. In particular, light is a very important aspect on the photosynthesis for fixing carbon dioxide (CO₂).

In particular, the lid 130 is formed of a material capable of transmitting light through the culture water bath 110, preferably without reflecting an artificial light source such as sunlight or a light-emitting diode (LED).

For this purpose, according to an exemplary embodiment of the present invention, the lid 130 is formed of polycarbonate. However, the present invention is not particularly limited thereto. Any materials, such as a plastic material, may be used as long as they can prevent the flow of a gas, such as carbon dioxide (CO₂), and transmit light.

The microalgae absorb carbon dioxide (CO₂) into culture water during photosynthesis of microalgae, and discharges oxygen (O₂) out of the culture water during respiration of microalgae. Therefore, in order to enhance the CO₂ fixation efficiency, the culture water bath 110 is hermetically sealed during the photosynthesis of microalgae, and the culture water bath 110 is unsealed during respiration of microalgae.

For this purpose, according to the present invention, a hydraulic pressure in the aquatic plant 120 may be used to facilitate hermetical sealing of the carbon dioxide (CO₂) and discharging of the oxygen (O₂).

As shown in FIG. 3, a watercourse 150 may be formed below the culture water bath 110 and the aquatic plant 120 so as to transfer the hydraulic pressure in the aquatic plant 120 to the inside of the culture water bath 110. In this case, the watercourse 150 may be formed of cement, and a surface of the watercourse may be finished with fiber-reinforced plastics (FRP). The FRP is a plastic material, and thus is basically light in weight, has low thermal conductivity and exhibits excellent characteristics such as durability, impact resistance, wear resistance, tensile strength, etc. Of course, the materials for forming the watercourse 150 are not limited to these specific materials, and other materials providing similar properties as cement and FRP may be used.

According to an embodiment of the present invention, the carbon dioxide (CO₂) present in the culture water bath is hermetically sealed under a hydraulic pressure during the photosynthesis of microalgae by adjusting a hydraulic level of water in the aquatic plant 120. In particular, the hydraulic level of water in the aquatic plant 120 can be set to a level higher than a hydraulic level of the culture water in the culture water bath 110 during photosynthesis.

On the other hand, the oxygen (O₂) formed in the culture water bath may be discharged out of the culture water bath 110 during the respiration of microalgae by adjusting a hydraulic level of water in the aquatic plant 120. In particular, the hydraulic level of water in the aquatic plant 120 can be set to a level lower than the hydraulic level of the culture water in the culture water bath 110 during respiration.

For this purpose, the aquatic plant 120 may have inlet holes (not shown) or other means of fluid communication formed therein for allowing water to flow in or out, and the hydraulic level of the aquatic plant 120 may be controlled by suitable control means, such as electric equipment equipped with an electric motor such as a water pump.

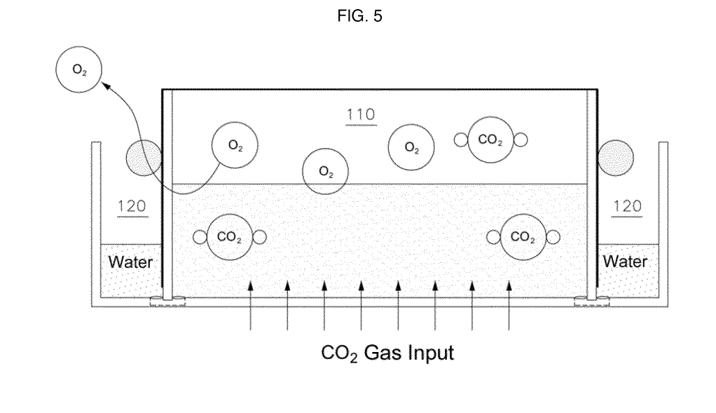

FIGS. 4 and 5 are exemplary diagrams illustrating a CO₂ sealing structure and an O₂ discharging structure of the photobioreactor for culturing microalgae according to one exemplary embodiment of the present invention.

As shown, the depicted embodiment of the present invention adopts a watercourse-type culture system. For example, the lid 130 may be formed of polycarbonate. The low solubility of carbon dioxide (CO₂) in water and a difference in hydraulic level (hydraulic pressure) may be used to induce hermetical sealing of the carbon dioxide (CO₂) using water as a sealing finish material. Discharging of gases in the culture system may be induced during the discharge of oxygen (O₂) formed by the photosynthesis of microalgae by lowering a hydraulic level of water hermetically sealed in the culture system to a hydraulic level of microalgae culture water.

FIG. 4 shows that carbon dioxide (CO₂) is hermetically sealed during the photosynthesis of microalgae. Here, the carbon dioxide (CO₂) is completely sealed using a difference in pressure caused by increasing a hydraulic level of water hermetically sealed in the aquatic plant 120 to a level greater than that of the culture water in the culture water bath 110.

FIG. 5 shows that oxygen (O₂) is discharged during the respiration of microalgae. Here, the oxygen (O₂) is discharged out using a difference in pressure caused by lowering a hydraulic level of water hermetically sealed in the aquatic plant 120 to a level greater than that of the culture water in the culture water bath 110.

The CO₂ sealing rates, the conversion yields and the manufacturing costs of the photobioreactor for culturing microalgae according to the present invention as formed as described above, the conventional watercourse-type culture system and the closed system are listed in the following Table 1.

TABLE 1

| | | Tubular | Watercourse- | |
|---|---|---|---|---|
| | | culture | type culture | Present |
| Improvement items | | system | system | Invention |
| Performance | CO₂ Sealing | 100% | 0% | 95% |
| improvement | rate | | | |
| | CO₂ | 55.6% | 35% | 55% |
| | Conversion | | | |
| | yield | | | |
| Cost cutting | Investment | 40,000 Won/ | 11,000 Won/ | 20,000 Won/m |
| | cost | m | m | |

As shown in Table 1, the photobioreactor for culturing microalgae according to the present invention had a good CO₂ sealing rate substantially comparable with that of the conventional tubular culture system, and a CO₂ conversion yield substantially identical to that of the tubular culture system.

For reference, the carbon dioxide conversion yield refers to a value obtained by dividing the biomass output of microalgae by the CO₂ input. In the present invention, it was confirmed that the CO₂ conversion yield was increased by a level of 20%, compared with that of the conventional watercourse-type culture system.

Also, it could be seen that the photobioreactor for culturing microalgae according to the present invention was installed substantially at halt the installation cost, compared with the manufacturing cost of the conventional tubular culture system.

As described above, the photobioreactor for culturing microalgae according to the present invention can realize complete CO₂ sealing, and high fixation efficiencies with a relatively low manufacturing cost by the present configuration which includes a mounting lid, a roller and an aquatic plant as described to provide a closed system during photosynthesis and an open system during respiration.

Therefore, the photobioreactor for culturing microalgae according to the present invention can be useful to improve the CO₂ fixation efficiency and CO₂ conversion yield, compared with the conventional watercourse-type culture system.

According to the present invention, the installation cost can be significantly reduced, compared with the conventional tubular culture system.

The present invention has been described in detail with reference to preferred embodiments thereof. However, it will be appreciated by those skilled in the art that changes may be made in these embodiments without departing from the principles of the invention, the scope of which is defined in the appended claims and equivalents thereof.

What is claimed is:

1. A photobioreactor for culturing microalgae, comprising:
a culture water bath configured to store culture water containing microalgae and having at least one inlet for feeding carbon dioxide (CO₂) into the culture water;
an aquatic plant formed on at least one side of the culture water bath to store water (H₂O); and
a lid configured to cover an upper portion of the culture water bath,
wherein, a watercourse is formed below the culture water bath and the aquatic plant.

2. The photobioreactor for culturing microalgae of claim 1, wherein the lid is formed of polycarbonate.

3. The photobioreactor for culturing microalgae of claim 1, wherein the lid extends along a wall frame forming the culture water bath.

4. The photobioreactor for culturing microalgae of claim 1, further comprising:
at least one roller formed on a side of the culture water bath to closely attach the lid to the culture water bath.

5. The photobioreactor for culturing microalgae of claim 1, wherein the watercourse is formed of cement, and a surface of the watercourse is finished with fiber-reinforced plastics (FRP).

6. The photobioreactor for culturing microalgae of claim 1, wherein the carbon dioxide (CO₂) present in the culture water bath is hermetically sealed under a hydraulic pressure by adjusting a hydraulic level of water in the aquatic plant so that the hydraulic level of water in the aquatic plant is set to a level higher than a hydraulic level of the culture water in the culture water bath.

7. The photobioreactor for culturing microalgae of claim 1, wherein the oxygen (O₂) formed in the culture water bath is discharged out of the culture water bath by adjusting a hydraulic level of water in the aquatic plant so that the hydraulic level of water in the aquatic plant is set to a level lower than the hydraulic level of the culture water in the culture water bath.
